(19) United States
(12) Patent Application Publication
(10) Pub. No.: US 2018/0117123 A1
(43) Pub. Date: May 3, 2018

(54) EXENATIDE-CONTAINING COMPOSITION AND PREPARATION METHOD THEREOF

(71) Applicant: SHANDONG LUYE PHARMACEUTICAL CO., LTD., Yantai (CN)

(72) Inventors: Tao Wang, Yantai (CN); Qilin Wang, Yantai (CN); Qianqian Xu, Yantai (CN); Lifang Sun, Yantai (CN); Ying Meng, Yantai (CN); Ju Li, Yantai (CN)

(73) Assignee: SHANDONG LUYE PHARMACEUTICAL CO., LTD., Yantai (CN)

(21) Appl. No.: 15/803,807

(22) Filed: Nov. 5, 2017

Related U.S. Application Data
(63) Continuation of application No. 15/304,528, filed on Oct. 16, 2016.

(30) Foreign Application Priority Data
Apr. 16, 2014 (CN) 201410153089.5
Apr. 16, 2014 (CN) 201410153581.1

(51) Int. Cl. A61K 38/26; A61K 9/16; A61K 38/22; A61K 47/34

(52) U.S. Cl. CPC A61K 38/26; A61K 9/1682; A61K 9/1647; A61K 9/16; A61K 47/34; A61K 38/22

(57) ABSTRACT

An exenatide-containing composition is in the form of microspheres, which are prepared by using exenatide acetate and polylactic-co-glycolic acid (PLGA) as raw materials, and the content of acetic acid in the prepared composition is lower than 0.01%. A method for preparing the exenatide-containing microspheres includes selecting a cleaning fluid of a proper external water phase and a proper amount to clean microspheres, and selecting a proper re-drying temperature to cure the microspheres after the microspheres are freeze-dried.

### CROSS REFERENCE TO RELATED APPLICATIONS

This is a continuation application of U.S. application Ser. No. 15/304,528, filed on Oct. 16, 2016 as a national stage application of PCT/CN2015/076688, filed on Apr. 16, 2015, which claims the priority of Chinese Application No. 201410153089.5, filed on Apr. 16, 2014, and Chinese Application No. 201410153581.1, filed on Apr. 16, 2014. This application claims the benefits and priority of all these prior applications and incorporates their disclosures by reference in their entirety.

### TECHNICAL FIELD

The present invention relates to pharmaceuticals, more particularly, to an exenatide-containing composition, which is in the form of microspheres. The present invention also relates to methods for preparing the same.

### BACKGROUND

Exenatide is the first synthesized glucagon-like peptide-1 (GLP-1) which has been used as an incretin analogue. It may be released from intestine into the circulatory system and leads to enhancement of glucose-dependent insulin secretion by simulating the way GLP-1 secreting in the normal physiological state of human. Exenatide shares partial amino acid sequence identity with mammalian GLP-1. Several in vivo study results have shown that exenatide could bind and activate the known human GLP-1 receptors to promote the synthesis of glucose-dependent insulin and the secretion of insulin in the pancreatic beta cells though cAMP and/or other intracellular signal transduction pathway. Under the conditions of elevated blood glucose concentrations, exenatide can improve glucose control through both promoting release of insulin in pancreatic beta cells and reducing blood glucose levels of fasting blood glucose and postprandial blood glucose at the same time.

Exenatide injections are the main approved exenatide products, and only used for delivery of subcutaneous injection. The initial dose is recommended as 5 μg twice a day for one month, then the dose may be increased to 105 μg based on the clinical responses. Pharmaceutical preparations are usually given by injection twice a day, and mainly eliminated by kidney. Its average peak time is 2.1 h, and the average elimination half-life is 2.4 h. The medical compliance of the diabetics who need long term administration is poor because of the frequent injection, and the injections cannot continuously activate the GLP-1 receptors.

Phase separation method has already been used as a preparation method of exenatide-containing microspheres in several prior arts. Phase separation method consists of many steps and needs high-level equipment and complex process. The microspheres products prepared by phase separation method need to be disposed through several processes to meet pharmaceutical products related quality standard requirements, for example sterile requirement and solvent residue requirement. To overcome the afore-mentioned deficiencies, several institutes have used solvent evaporation technique as the preparation method of exenatide-containing microspheres, finding that the technique consists of less steps, needs lower level equipment, and less processes needed to dispose the microspheres, as compared with phase separation method. The ingredient of the approved exenatide products is exenatide acetate. The standard content of acetic acid in the microspheres is equal or greater than 3% and less than or equal to 12%, while no control requirement of the content of acetic acid during the manufacturing process of the exenatide-containing microspheres has been mentioned in the prior art. Some studies have indicated that the contents of acetic acid during the manufacturing process of exenatide-containing microspheres has a great effect on the bioavailability of the final products within storage period, and the bioavailability of exenatide within the product storage period will decline when the content of acetic acid is above 0.01%.

### SUMMARY

To solve the technical defects existing in the prior art, inventors of the present invention provide exenatide-containing compositions through in-depth research, and the composition is in the form of microspheres. The exenatide-containing microspheres comprise exenatide acetate (approved product, 3%≤the content of acetic acid ≤12%) and copolymers of lactide and glycolide (PLGA) as raw materials, and the contents of acetic acid in the prepared microspheres are below 0.01%.

The present invention provides exenatide-containing compositions. The compositions are in the form of microspheres. The exenatide-containing microspheres comprise exenatide acetate and copolymers of lactide and glycolide (PLGA) as starting materials. The contents of acetic acid in the prepared compositions are below 0.01%.

In a composition of the present invention, the content of exenatide is 2-10% by weight, preferably 3-8%, and more preferably 4-6%. The content of the copolymer of lactide and glycolide is 90-98% by weight, preferably 92-97%, and more preferably 94-96%.

The copolymer of lactide and glycolide is also referred to as poly (lactide-co-glycolide), abbreviated as PLGA. The PLGA described in the present invention has a molecular weight of 6,000-45,000 Dalton, preferably 10,000-30,000 Dalton, and more preferably 10,000-25,000 Dalton. As used herein, the term “molecular weight” refers to “weight average molecular weight,” which is referred to herein as “molecular weight.” The molar ratio of lactide to glycolide in said PLGA is from 90:10 to 10:90, preferably from 75:25 to 25:75, more preferably from 60:40 to 40:60, and especially 50:50. The intrinsic viscosity of PLGA is 0.10-0.40 dL/g, preferably in the range of 0.10-0.35 dL/g, and optimally in the range of 0.10-0.30 dL/g. A method for measuring the intrinsic viscosity of PLGA is as follows: prepare an about 0.5% (w/v) solution of PLGA in chloroform, and determine the intrinsic viscosity of PLGA at 30° C. using a Cannon-Fenske glass capillary viscometer.

For convenience of description, the molar ratio of lactide to glycolide, the intrinsic viscosity and molecular weight of PLGA are shown hereinafter in brackets following each PLGA. For example, “PLGA (50/50, 0.20, 16,000)” represents poly (lactide-co-glycolide) with a molar ratio of lactide to glycolide of 50:50, an intrinsic viscosity of 0.20 dL/g, and a molecular weight of 16,000 Dalton.

The compositions provided by the present invention are prepared by solid-in-oil-in-water (s/o/w) emulsion-solvent evaporation process. Therefore, the present invention also provides methods for preparing the exenatide-containing compositions.

The microspheres provided by the present invention are prepared as follows: PLGA is dissolved in an organic solvent to form an organic solution; exenatide acetate is added into the organic solution to obtain a primary emulsion. Then, the primary emulsion is added into an outer aqueous phase and emulsified to obtain an emulsion. Then, the emulsion is solidified and volatilized to remove the organic solvent. The residue was washed and freeze-dried to obtain the freeze-dried microspheres.

The organic solvents described in the present disclosure may be the organic solvents into which the PLGA could be dissolved, including poly (lactide-co-glycolide) (PLGA), dichloromethane, acetone, acetonitrile, etc., preferably dichloromethane.

In the manufacturing process of a composition provided by the present invention, a cleaning solution in an appropriate proportion may be used, and a specified re-drying temperature used for freeze-drying microspheres may be set at the same time. The cleaning solution is selected from distilled water or an alkaline buffer solution, preferably distilled water. The proportion by weight of the microspheres and the cleaning solution is 1:250, preferably 1:300 and above. The specified re-drying temperature of freeze-dried microspheres is set lower than the glass-transition temperature of the mixture of exenatide acetate and PLGA. A composition of exenatide-containing microspheres with good characteristic could be obtained through the aforesaid adjusting, which contains a very low amount of acetic acid and meets the quality standard.

In the manufacturing process of a composition provided by the present invention, the concentration and pH value of outer aqueous phase may be adjusted. The outer aqueous phase is a polyvinyl alcohol solution, and the average molecular weight of the polyvinyl alcohol is 13,000-23,000. In the polyvinyl alcohol solution, the content by volume of polyvinyl alcohol is 0.5-1.5%, preferably 1%. The PH value of the polyvinyl alcohol solution is 5.6±1.0, preferably 5.6±0.5, and more preferably 5.6±0.2. In the composition provided in the present disclosure, the content of polyvinyl alcohol residue should not be more than 0 008% (the content by weight).

Specifically, the microspheres provided by the present invention may be prepared by the solvent evaporation process as follows: a certain amount of exenatide or a salt or an analogue thereof is weighed and dissolved in distilled water to form an aqueous solution. Known amounts of excipients are weighed and dissolved in the aqueous solution. The excipients may include a co-solvent, a protein protectant, a surface active agent, and a pore forming agent, etc. A certain amount of PLGA is weighed and dissolved in an organic solvent in which PLGA is soluble. The organic solvent may be selected from dichloromethane, acetone, acetonitrile, etc., preferably dichloromethane The PLGA organic solution is then mixed with the aqueous solution containing exenatide and excipients to form a primary emulsion. The primary emulsion containing exenatide or a salt or an analogue thereof is added into an outer aqueous phase (e.g., a polyvinyl alcohol solution) and emulsified to obtain an emulsion. Then, the emulsion is solidified and volatilized to remove the organic solvent. The residue was washed and freeze-dried to obtain the microspheres.

In the aforesaid manufacturing process, exenatide acetate need not be pre-prepared to form an aqueous solution. Exenatide acetate may be mixed directly with the PLGA organic solution to form a primary emulsion.

In a manufacturing process of the microspheres provided by the present invention, the outer aqueous phase is a polyvinyl alcohol solution. In the polyvinyl alcohol solution, the content by volume of polyvinyl alcohol is 0.5-1.5%, preferably 1%. The pH value of the solution is 5.6±1.0, preferably 5.6±0.5, and more preferably 5.6±0.2.

The pH value of the outer aqueous phase plays a key role for the characteristics of the microspheres provided by the present invention. In the present invention, the pH value of an outer aqueous phase may be adjusted by an acidic aqueous solution, a basic aqueous solution, or a buffer solution. The oil phase containing exenatide or a salt or an analogue thereof described in the present invention is emulsified to obtain an emulsion, and then the emulsion may be solidified in the outer aqueous phase whose pH value has been specified to form substrates of the microspheres with good characteristics.

The pH value of the outer aqueous phase can be kept stable within a certain range by adjusting with an acid aqueous solution, base aqueous solution, or a buffer solution. The acid should be a water-soluble acid, may be selected from hydrochloric acid, sulfuric acid, acetic acid and amino acid, and 0.1 mol/L hydrochloric acid aqueous solution is in common use. The base should be a water-soluble base, may be selected from sodium hydroxide, potassium hydroxide, sodium bicarbonate and sodium sulfite, and 0.1 mol/L sodium hydroxide aqueous solution is in common use. The buffer solution may be selected from citrate buffer solution, phosphate buffer solution, acetate buffer solution, and phthalate buffer solution, phthalate buffer solution (pH=5.6) is in common use.

The afore-mentioned acid aqueous solution, base aqueous solution and buffer solution may be prepared in accordance with the appendix of Chinese Pharmacopeia (second part, 2010 Edition). For example, 0.1 mol/L hydrochloric acid aqueous solution and 0.1 mol/L sodium hydroxide aqueous solution may be prepared in accordance with the preparation method of titrant recorded in appendix XV F of Chinese Pharmacopeia, and phthalate buffer solution (pH=5.6) may be prepared in accordance with the preparation method of titrant recorded in appendix XV D of Chinese Pharmacopeia.

In the manufacturing process of the microspheres described in the present invention, the content by volume of polyvinyl alcohol (g/100 ml) in the outer aqueous phase plays a key role in the characteristics of the microspheres. In the present invention, the content by volume of polyvinyl alcohol is 0.5-1.5%, preferably 1%. A composition of exenatide-containing microspheres with good characteristics may be obtained by solidifying the microspheres in the outer aqueous phase of a selected concentration.

Polyvinyl alcohol used in the present invention may have an average molecular weight of 13,000-23,000 Dalton. A solution of which plays an important role for the characteristics of the prepared microspheres. Exenatide-containing microspheres with good characteristics may be obtained by solidifying the microspheres in an outer aqueous phase comprising polyvinyl alcohol which has a selected average molecular weight.

In the manufacturing process of the exenatide-containing microspheres provided by the present disclosure, microspheres with good characteristics may be obtained by adjusting the polyvinyl alcohol concentration of the outer aqueous phase, wherein the characteristics including high entrapment efficiency, uniform particle sizes, and minimum residue of the polyvinyl alcohol.

In a manufacturing process of the exenatide-containing microspheres of the present invention, the amount of cleaning solution, the freeze-drying time and the temperature all play key roles in reducing the amount of acetic acid in the prepared microspheres.

The microspheres of the present invention may be cleaned by a cleaning solution, and the cleaning solution may be distilled water or a buffer solution. The proportion by weight of the microspheres and the cleaning solution is 1:250 and above, preferably 1:330 and above. The microspheres cleaned by the cleaning solution of selected proportion by weight of the microspheres and the cleaning solution can form substrates of the exenatide-containing microspheres having minimum residue of acetic acid.

PH value of the cleaning solution used to clean the microspheres provided in the present disclosure can be kept stable within a certain range by adjusting with distilled water or an alkaline buffer solution, preferably distilled water. The alkaline buffer solution may be selected from citrate buffer solution, phosphate buffer solution, acetate buffer solution, or phthalate buffer solution.

In the manufacturing process of the exenatide-containing microspheres of the present invention, the freeze-drying process consists of a pre-freezing procedure, a sublimation drying procedure, repeated freezing and sublimating procedure, and re-drying procedure. The freeze-drying procedure is a drying method in which the solution freezes into a solid, then the solid sublimates directly without becoming liquid state under low temperature and low pressure. In a freeze-drying process of the present invention, the re-drying temperature is specified, while the other procedures apply to the conventional methods.

In the freeze-drying process described in the present invention, when the sublimation drying procedure is completed, the re-drying procedure may be used to further remove the residual liquid as much as possible. In the freeze-drying process described in the present invention, the re-drying temperature plays an important role for the content of acetic acid containing in the prepared microspheres. The re-drying temperature used in the present invention is lower than the glass-transition temperature of the mixture of exenatide acetate and PLGA (emulsified) so as to obtain a composition of exenatide-containing microspheres with good characteristics in accordance with the quality standard.

In the manufacturing process of the exenatide-containing microspheres provided in the present invention, the contents of acetic acid may be controlled to extend the product shelf life of the composition and to maintain the bioavailability of the compositions unchanged during a long storage period.

In the manufacturing process of the microspheres provided in the present invention, exenatide acetate is used as the raw material, and the concentration of acetic acid is reduced to an extremely low amount to improve the stability of the prepared microspheres and to extend the product shelf lives of the compositions. The concentration of acetic acid is controlled by selecting of the amount of the cleaning solution, the freeze-drying time and the freeze-drying temperature for the formed microspheres. In the manufacturing process, the microspheres with good characteristics can be obtained by adjusting the concentration and the pH value of the outer aqueous phase, wherein the characteristics including high entrapment efficiency, uniform particle size and minimum residue of the polyvinyl alcohol.

### DETAILED DESCRIPTION

The present invention will be further illustrated with the following examples, which should not limit the scope of the present invention in any way.

### Example 1

The microspheres of exenatide or a salt or an analogue thereof are prepared in the outer aqueous phase of 0.5% polyvinyl alcohol aqueous solution (pH=6.5).

Preparation of the polyvinyl alcohol aqueous solution (PVA aqueous solution): an appropriate amount of solid polyvinyl alcohol (average molecular weight of 13000-23000 Dalton) and a calculated volume of water were weighed. Then, the solid polyvinyl alcohol was dissolved in half of the water, and the other half of water was not added until the solid polyvinyl alcohol had dissolved completely. The PVA aqueous solution was stirred until mixed thoroughly, and then its pH value was measured. The pH value of the PVA aqueous solution may be adjusted by adding 0.1 mol/L hydrochloric acid aqueous solution or 0.1 mol/L sodium hydroxide aqueous solution until it stabilizes at 6.5.

Preparation method: a certain amount of exenatide specified in the formulation was weighed and dissolved in distilled water under stirring, so as to obtain a clear solution. A certain amount of copolymers of lactide and glycolide (PLGA) specified in the formulation was weighed and dissolved in dichloromethane (CH₂Cl₂) under stirring, so as to obtain a clear solution. The exenatide aqueous solution was added into the PLGA solution to obtain a primary emulsion. The prepared PVA aqueous solution was subjected to sterile filtration, then added into a vacuum emulsification blender (referred herein as microspheres preparation kettle) to be cooled to 7-13° C. to obtain an outer aqueous phase. The primary emulsion was added into the microspheres preparation kettle under homogenization speed of 400 rpm, then it was homogeneously emulsified for 60 s. Then, it was emulsified under a reduced homogenization speed of 150 rpm for 5 h to obtain solid microspheres, which were then filtered and collected. Then, the microspheres are rinsed with water (see example 11 for the detailed method) to remove the PVA residue. The microspheres are then transferred into a freeze-drying tray, and mannitol and a proper amount of water for injection were then added therein. The freeze-drying tray was placed in a freeze drier for freeze-drying (see example 11 for the detailed method). The freeze-dried product was subjected to sieving and mixing to obtain the microspheres.

See tables 1-3 for the detailed measuring method of entrapment efficiency, particle sizes and polymer residue.

### Example 2

The microspheres of exenatide or a salt or an analogue thereof are prepared in the outer aqueous phase of 0.8% polyvinyl alcohol aqueous solution (pH=5.1).

Preparation of the polyvinyl alcohol aqueous solution (PVA aqueous solution): an appropriate amount of solid polyvinyl alcohol (average molecular weight of 13000-23000 Dalton) and a calculated volume of water were weighed. Then, the solid polyvinyl alcohol was dissolved in half of the water, and the other half of water was not added until the solid polyvinyl alcohol had dissolved completely. The PVA aqueous solution was stirred until mixed evenly, and then its pH value was measured. The pH value of the PVA aqueous solution could be adjusted by adding 0.1 mol/L hydrochloric acid aqueous solution or 0.1 mol/L sodium hydroxide aqueous solution until it stabilizes at 5.1.

Preparation method: preparing the microspheres as described in example 1.

See tables 1-3 for the detailed measuring method of entrapment efficiency, particle sizes and polymer residue.

### Example 3

The microspheres of exenatide or a salt or an analogue thereof are prepared in the outer aqueous phase of 1.0% polyvinyl alcohol aqueous solution (pH=5.6).

Preparation of the polyvinyl alcohol aqueous solution (PVA aqueous solution): an appropriate amount of solid polyvinyl alcohol (average molecular weight of 13000-23000 Dalton) and a calculated volume of water were weighed. Then, the solid polyvinyl alcohol was dissolved in half of the water, and the other half of water was not added until the solid polyvinyl alcohol had dissolved completely. The PVA aqueous solution was stirred until mixed evenly, and then its pH value was measured. The pH value of the PVA aqueous solution could be adjusted by adding phthalate buffer until it stabilizes at 5.6.

Preparation method: preparing the microspheres as described in example 1.

See tables 1-3 for the detailed measuring method of entrapment efficiency, particle sizes and polymer residue.

### Example 4

The microspheres of exenatide or a salt or an analogue thereof are prepared in the outer aqueous phase of 1.2% polyvinyl alcohol aqueous solution (pH=6.1).

Preparation of the polyvinyl alcohol aqueous solution (PVA aqueous solution): an appropriate amount of solid polyvinyl alcohol (average molecular weight of 13000-23000 Dalton) and a calculated volume of water were weighed. Then, the solid polyvinyl alcohol was dissolved in half of the water, and the other half of water was not added until the solid polyvinyl alcohol had dissolved completely. The PVA aqueous solution was stirred until mixed evenly, and then its pH value was measured. The pH value of the PVA aqueous solution could be adjusted by adding 0.1 mol/L hydrochloric acid aqueous solution or 0.1 mol/L sodium hydroxide aqueous solution until it stabilizes at 6.1.

Preparation method: preparing the microspheres as described in example 1.

See tables 1-3 for the detailed measuring method of entrapment efficiency, particle sizes and polymer residue.

### Example 5

The microspheres of exenatide or a salt or an analogue thereof are prepared in the outer aqueous phase of 1.5% polyvinyl alcohol aqueous solution (pH=4.6).

Preparation of the polyvinyl alcohol aqueous solution (PVA aqueous solution): an appropriate amount of solid polyvinyl alcohol (average molecular weight of 13000-23000 Dalton) and a calculated volume of water were weighed. Then, the solid polyvinyl alcohol was dissolved in half of the water, and the other half of water was not added until the solid polyvinyl alcohol had dissolved completely. The PVA aqueous solution was stirred until mixed evenly, and then its pH value was measured. The pH value of the PVA aqueous solution could be adjusted by adding 0.1 mol/L hydrochloric acid aqueous solution or 0.1 mol/L sodium hydroxide aqueous solution until it stabilizes at 4.6.

Preparation method: preparing the microspheres as described in example 1.

See tables 1-3 for the detailed measuring method of entrapment efficiency, particle sizes and polymer residue.

### Example 6

The microspheres of exenatide or a salt or an analogue thereof are prepared in the outer aqueous phase of 1.0% polyvinyl alcohol aqueous solution (pH=6.5).

Preparation of the polyvinyl alcohol aqueous solution (PVA aqueous solution): an appropriate amount of solid polyvinyl alcohol (average molecular weight of 13000-23000 Dalton) and a calculated volume of water were weighed. Then, the solid polyvinyl alcohol was dissolved in half of the water, and the other half of water was not added until the solid polyvinyl alcohol had dissolved completely. The PVA aqueous solution was stirred until mixed evenly, and then its pH value was measured. The pH value of the PVA aqueous solution could be adjusted by adding 0.1 mol/L hydrochloric acid aqueous solution or 0.1 mol/L sodium hydroxide aqueous solution until it stabilizes at 6.5.

Preparation method: preparing the microspheres as described in example 1.

See tables 1-3 for the detailed measuring method of entrapment efficiency, particle sizes and polymer residue.

### Example 7

The microspheres of exenatide or a salt or an analogue thereof are prepared in the outer aqueous phase of 1.0% polyvinyl alcohol aqueous solution (pH=4.7).

Preparation of the polyvinyl alcohol aqueous solution (PVA aqueous solution): an appropriate amount of solid polyvinyl alcohol (average molecular weight of 13000-23000 Dalton) and a calculated volume of water were weighed. Then, the solid polyvinyl alcohol was dissolved in half of the water, and the other half of water was not added until the solid polyvinyl alcohol had dissolved completely. The PVA aqueous solution was stirred until mixed evenly, and then its pH value was measured. The pH value of the PVA aqueous solution could be adjusted by adding 0.1 mol/L hydrochloric acid aqueous solution or 0.1 mol/L sodium hydroxide aqueous solution until it stabilizes at 4.7.

Preparation method: preparing the microspheres as described in example 1.

See tables 1-3 for the detailed measuring method of entrapment efficiency, particle sizes and polymer residue.

### Example 8

The microspheres of exenatide or a salt or an analogue thereof are prepared in the outer aqueous phase of 1.0% polyvinyl alcohol aqueous solution (pH=5.6).

Preparation of the polyvinyl alcohol aqueous solution (PVA aqueous solution): an appropriate amount of solid polyvinyl alcohol (average molecular weight of 13000-23000 Dalton) and a calculated volume of water were weighed. Then, the solid polyvinyl alcohol was dissolved in half of the water, and the other half of water was not added until the solid polyvinyl alcohol had dissolved completely. The PVA aqueous solution was stirred until mixed evenly, and then its pH value was measured. The pH value of the PVA aqueous solution could be adjusted by adding phthalate buffer until it stabilizes at 5.6.

Preparation method: preparing the microspheres as described in example 1.

See tables 1-3 for the detailed measuring method of entrapment efficiency, particle sizes and polymer residue.

### Comparative Example 1

The microspheres of exenatide or a salt or an analogue thereof are prepared in the outer aqueous phase of 1.0% polyvinyl alcohol aqueous solution (pH=9.0).

Preparation of the polyvinyl alcohol aqueous solution (PVA aqueous solution): an appropriate amount of solid polyvinyl alcohol (average molecular weight of 13000-23000 Dalton) and a calculated volume of water were weighed. Then, the solid polyvinyl alcohol was dissolved in half of the water, and the other half of water was not added until the solid polyvinyl alcohol had dissolved completely. The PVA aqueous solution was stirred until mixed evenly, and then its pH value was measured. The pH value of the PVA aqueous solution could be adjusted by adding 0.1 mol/L hydrochloric acid aqueous solution or 0.1 mol/L sodium hydroxide aqueous solution until it stabilizes at 9.0.

Preparation method: preparing the microspheres as described in example 1.

See tables 1-3 for the detailed measuring method of entrapment efficiency, particle sizes and polymer residue.

### Comparative Example 2

The microspheres of exenatide or a salt or an analogue thereof are prepared in the outer aqueous phase of 1.0% polyvinyl alcohol aqueous solution (pH=3.0).

Preparation of the polyvinyl alcohol aqueous solution (PVA aqueous solution): an appropriate amount of solid polyvinyl alcohol (average molecular weight of 13000-23000 Dalton) and a calculated volume of water were weighed. Then, the solid polyvinyl alcohol was dissolved in half of the water, and the other half of water was not added until the solid polyvinyl alcohol had dissolved completely. The PVA aqueous solution was stirred until mixed evenly, and then its pH value was measured. The pH value of the PVA aqueous solution could be adjusted by adding 0.1 mol/L hydrochloric acid aqueous solution or 0.1 mol/L sodium hydroxide aqueous solution until it stabilizes at 3.0.

Preparation method: preparing the microspheres as described in example 1.

See tables 1-3 for the detailed measuring method of entrapment efficiency, particle sizes and polymer residue.

### Comparative example 3

The microspheres of exenatide or a salt or an analogue thereof are prepared in the outer aqueous phase of 5.0% polyvinyl alcohol aqueous solution (pH=5.8).

Preparation of the polyvinyl alcohol aqueous solution (PVA aqueous solution): an appropriate amount of solid polyvinyl alcohol (average molecular weight of 13000-23000 Dalton) and a calculated volume of water were weighed. Then, the solid polyvinyl alcohol was dissolved in half of the water, and the other half of water was not added until the solid polyvinyl alcohol had dissolved completely. The PVA aqueous solution was stirred until mixed evenly, and then its pH value was measured. The pH value of the PVA aqueous solution could be adjusted by adding 0.1 mol/L hydrochloric acid aqueous solution or 0.1 mol/L sodium hydroxide aqueous solution until it stabilizes at 5.8.

Preparation method: preparing the microspheres as described in example 1.

See tables 1-3 for the detailed measuring method of entrapment efficiency, particle sizes and polymer residue.

### Comparative Example 4

The microspheres of exenatide or a salt or an analogue thereof are prepared in the outer aqueous phase of 0.1% polyvinyl alcohol aqueous solution (pH=5.6).

Preparation of the polyvinyl alcohol aqueous solution (PVA aqueous solution): an appropriate amount of solid polyvinyl alcohol (average molecular weight of 13000-23000 Dalton) and a calculated volume of water were weighed. Then, the solid polyvinyl alcohol was dissolved in half of the water, and the other half of water was not added until the solid polyvinyl alcohol had dissolved completely. The PVA aqueous solution was stirred until mixed evenly, and then its pH value was measured. The pH value of the PVA aqueous solution could be adjusted by adding 0.1 mol/L hydrochloric acid aqueous solution or 0.1 mol/L sodium hydroxide aqueous solution until it stabilizes at 5.6.

Preparation method: preparing the microspheres as described in example 1.

See tables 1-3 for the detailed measuring method of entrapment efficiency, particle sizes and polymer residue.

### Test Example 1

### Measurement of Entrapment Efficiency for the Microspheres of Exenatide or a Salt or an Analogue Thereof

The entrapment efficiency of the microspheres of exenatide or a salt or an analogue thereof may be determined as follows: 20 mg microspheres prepared in the examples were weighed and dissolved in 2 ml glacial acetic acid under ultrasonication in a 10 ml volumetric flask to obtain a clear and transparent solution. The solution was diluted with water to volume, and then mixed and allowed to stand until the insoluble substance settled. Then, a supernatant was obtained and used as a test solution. An appropriate amount of reference exenatide was precisely weighed and dissolved in water, and then diluted to obtain a reference solution having a concentration of about 0.1 mg/ml. A test solution (20 μl) and a reference solution (20 μl) were weighed, respectively. Then, both were injected into a liquid chromatograph. Record the chromatograms, and then determine the content of exenatide in the test solution using an external standard method based on the peak area.

The entrapment efficiency is expressed as % of the actual entrapment quantity relative to the theoretical quantity. See table 1 for the measurement results.

TABLE 1
Entrapment efficiency of the microspheres of exenatide or a salt or an
analogue thereof

| | Entrapment |
|---|---|
| Test NO. | efficiency |
| Example 1 | 85.4% |
| Example 2 | 91.6% |
| Example 3 | 99.1% |
| Example 4 | 90.3% |
| Example 5 | 88.3% |
| Example 6 | 93.1% |
| Example 7 | 92.5% |
| Example 8 | 85.8% |
| Comparative | 64.2% |
| example 1 | |
| Comparative | 69.1% |
| example 2 | |
| Comparative | 68.2% |
| example 3 | |
| Comparative | 66.4% |
| example 4 | |

Analysis of results: for the microspheres of exenatide or a salt or an analogue thereof prepared in examples 1-8, the entrapment efficiencies of the microspheres were not affected by methods of adding exenatide and solution, but were affected by the concentrations and the pH values of the outer aqueous phases. The entrapment efficiencies of the microspheres were always above 85% when the concentrations of the outer aqueous phases and the pH values were under controlled. In the comparative examples, the entrapment efficiencies of the microspheres were always below 70% when the concentrations and the pH values of the outer aqueous phases were out of the controlled range.

### Test Example 2

### Measurement of the Span for the Microspheres of Exenatide or a Salt or an Analogue Thereof

According to the related regulations on particle size and particle size distribution measurement method [The third method in Appendix XIX E of Chinese Pharmacopoeia (second part, 2010 Edition)], 0.1% Tween 20 solution was used as a dispersing agent. About 120 ml of Tween 20 solution was transferred into the sample dispersion device of a particle size analyzer (The Malvern Mastersizer 2000 Particle Size Analyzer), and the rotational speed controller was adjusted so that the stirring was at 2,100 rpm. The background of dispersing agent was measured first. An appropriate amount of exenatide-containing microspheres was added into the dispersing agent. After the sample was distributed evenly, the particle sizes D90, D50 and D10 were measured in parallel for three times and the average numbers were taken. The particle size distribution span used in the present invention is defined according to the Guidelines for Microcapsules, Microspheres and Liposome Preparations, Appendix XIXE of Chinese Pharmacopoeia (2010 Edition) as follows:

Span=(D90−D10)/D50

The span was calculated according to the above formula. See table 2 for the measurement results.

| Test NO. | Span |
|---|---|
| Example 1 | 0.76 |
| Example 2 | 0.74 |
| Example 3 | 0.24 |
| Example 4 | 0.78 |
| Example 5 | 0.64 |
| Example 6 | 0.44 |
| Example 7 | 0.41 |
| Example 8 | 0.79 |
| Comparative | 2.01 |
| example 1 | |
| Comparative | 2.29 |
| example 2 | |
| Comparative | 2.04 |
| example 3 | |
| Comparative | 2.07 |
| example 4 | |

Analysis of results: for the microspheres of exenatide or a salt or an analogue thereof prepared in Examples 1-8, the span of the microspheres were not affected by the addition methods of the exenatide and the solutions, but were affected by the concentrations and the pH values of the outer aqueous phases. The particle size distribution spans of the microspheres were always below 0.8, when the concentrations of the outer aqueous phases and the pH values were under control. According to the measured particle size distribution span results, the sizes of microspheres product were uniform. In the comparative examples, the particle size distribution spans of the microspheres were above 2.0, when the concentrations and the pH values of the outer aqueous phases were out of the controlled range, which showed that sizes of microspheres product were not uniform.

### Test Example 3

### Measurement of the Content of Polyvinyl Alcohol Residue in the Microspheres of Exenatide or a Salt or an Analogue Thereof

Determination method of the content of polyvinyl alcohol residue:
The chromatographic conditions were as follows:
Stationary phase: the gel chromatography column (TSK-GEL G2000SWXL)
Mobile phase: the potassium dihydrogen phosphate buffer solution pH 7.0
Flow rate: 0.8 ml/min.
The differential refractive index detector was used.

Determination of a standard curve: an appropriate amount of PVA was precisely weighted and used to prepare a 1.0 mg/ml PVA mother solution. Then, the mother solution was diluted to prepare a series of standard solutions. The diluted concentrations were 0.01, 0.02, 0.05, 0.1, 0.2 and 0.3 mg/ml, respectively. Ten (10) μl of each standard solution was injected into the liquid chromatograph. Record the chromatograms, and then the standard curve was established by sample concentration as X-axis and peak area as Y-axis. The linear regression coefficient was not lower than 0.999 in the linear regression model.

Measurement method: 500 mg of microspheres of the present invention were precisely weighed and degraded in 50 ml of 2 mol/L sodium hydroxide aqueous solution for 36 h. The solution was neutralized with 2 mol/L hydrochloric acid aqueous solution. The PLGA degradation products with molecular weight of 3500 Dalton may be removed by dialysis in a dialysis bag for 36 h to obtain a solution, and then the solution was vacuum freeze-dried to obtain the polyvinyl alcohol residue. The polyvinyl alcohol residue was re-dissolved and diluted to 2 ml to obtain the test solution. Hundred (100) μl test solution was injected into the liquid chromatograph. Then, record the chromatograms to calculate the contents of polyvinyl alcohol residues in microspheres using an external standard method.

Table 3 shows contents of polyvinyl alcohol residues in the microspheres of exenatide or a salt or an analogue thereof

| | polyvinyl alcohol |
|---|---|
| Test NO. | residue |
| Example 1 | 0.0067% |
| Example 2 | 0.0054% |
| Example 3 | 0.0019% |
| Example 4 | 0.0068% |
| Example 5 | 0.0079% |
| Example 6 | 0.0074% |
| Example 7 | 0.0043% |
| Example 8 | 0.0072% |
| Comparative | 0.0281% |
| example 1 | |
| Comparative | 0.0263% |
| example 2 | |
| Comparative | 0.0397% |
| example 3 | |
| Comparative | 0.0157% |
| example 4 | |

Analysis of results: for the microspheres of exenatide or a salt or an analogue thereof prepared in examples 1-8, the contents of polyvinyl alcohol residues in the microspheres were not affected by the concentrations and the pH values of the outer aqueous phases. The measured contents of polyvinyl alcohol residues in the microspheres prepared in examples 1-7 are below 0.008%. The clinical use is safe because there is only minimum content of polyvinyl alcohol residue in the microspheres. In the comparative examples, the contents of polyvinyl alcohol residues in the microspheres were above 0.01%, namely relatively high content.

### Example 9

Preparation of the Exenatide-containing Microspheres with Content (By Weight) of the (Exenatide Acetate+PLGA) and the Cleaning Solution of 1:250.

Preparation method: 7.5 g exenatide acetate was weighed and dissolved in 25 ml distilled water under stirring to obtain a solution. Dissolve 142.5 g of a copolymer of lactide and glycolide (PLGA 5050 2A 16000) in 675 ml dichloromethane (CH₂Cl₂) under stirring to obtain another solution. Then, the exenatide acetate solution was mixed with the PLGA solution to obtain a primary emulsion. Subject 75 L of the prepared 1% PVA aqueous solution (see example 3 for the detailed preparation method) to sterile filtration. It was then added into a vacuum emulsification blender (referred herein as microspheres preparation kettle) and cooled to 7-13° C. for use as an outer aqueous phase. The primary emulsion was added into the microspheres preparation kettle and homogenized at rate of 400 rpm. Then, it was homogeneously emulsified for 60 s, and then emulsified at a reduced homogenization rate of 150 rpm for 5 h to obtain solid microspheres, which then could be filtered and collected. The microspheres were rinsed with 37.5 g distilled water, and then transferred into a freeze-drying tray. An appropriate amount of mannitol injection solution and an appropriate amount of water were added into the freeze-drying tray, and then the freeze-drying tray was placed in a freeze drier to freeze-dry for 36 h at 40° C. (the glass-transition temperature of the mixture of exenatide acetate and PLGA is 43° C.). Then, the freeze-dried product was subjected to sieving and mixing to obtain the exenatide-containing micro spheres.

See table 4 for the determination method of the contents of acetic acid in the microspheres.

### Example 10

Preparation of the exenatide-containing microspheres with content (by weight) of the (exenatide acetate+PLGA) and the cleaning solution of 1:300.

Preparation method: Weigh 7.5 g of exenatide acetate and dissolve it in 25 ml distilled water under stirring to obtain a solution. Dissolve 142.5 g of a copolymer of lactide and glycolide (PLGA 5050 2A 16000) in 675 ml of dichloromethane (CH₂Cl₂) under stirring to obtain another solution. Then, the exenatide acetate solution was mixed with the PLGA solution to obtain a primary emulsion. Subject 75 L of the prepared 1% PVA aqueous solution (see example 3 for the detailed preparation method) to sterile filtration. Then, it was added into a vacuum emulsification blender (referred herein as microspheres preparation kettle) and cooled to 7-13° C. for use as an outer aqueous phase. The primary emulsion was added into the microspheres preparation kettle under a homogenization rate of 400 rpm. It was homogeneously emulsified for 60 s, and then it was emulsified under a reduced homogenization rate of 150 rpm for 4 h to obtain the solid microspheres, which were then filtered and collected. The microspheres were rinsed with 45 g distilled water, and then transferred into a freeze-drying tray. An appropriate amount of mannitol injection solution and an appropriate amount of water were added into the freeze-drying tray. Then, the freeze-drying tray was placed in a freeze drier to free dry for 36 h at 40° C. (the glass-transition temperature of the mixture of exenatide acetate and PLGA is 43° C.). Then, the freeze-dried product was subjected to sieving and mixing to obtain the microspheres.

See table 4 for the determination method for the contents of acetic acid in the microspheres.

### Example 11

Preparation of the exenatide-containing microspheres with content (by weight) of the (exenatide acetate+PLGA) and the cleaning solution of 1:330.

Preparation method: Weigh 7.5 g of exenatide acetate and dissolve it in 25 ml of distilled water under stirring to obtain a solution. Dissolve 142.5 g of a copolymer of lactide and glycolide (PLGA 5050 2A 16000) in 675 ml of dichloromethane (CH₂Cl₂) under stirring to obtain another solution. Then, the exenatide acetate solution was mixed with the PLGA solution to obtain a primary emulsion. Subject 75 L of the prepared 1% PVA aqueous solution (see example 3 for the detailed preparation method) to sterile filtration. Then, it was added into a vacuum emulsification blender (referred herein as microspheres preparation kettle) and cooled to 7-13° C. for use as an outer aqueous phase. The primary emulsion was added into the microspheres preparation kettle under a homogenization rate of 400 rpm. It was homogeneously emulsified for 60 s, and then it was emulsified under a reduced homogenization rate of 150 rpm for 4 h to obtain the solid microspheres, which were then filtered and collected. The microspheres were rinsed with 49.5 g of distilled water and then transferred into a freeze-drying tray. An appropriate amount of mannitol injection solution and an appropriate amount of water were added into the freeze-drying tray. Then, the freeze-drying tray was placed in a freeze drier to freeze dry for 36 h at 40° C. (the glass-transition temperature of the mixture of exenatide acetate and PLGA is 43° C.). Then, the freeze-dried product was subjected to sieving and mixing to obtain the microspheres.

See table 4 for the determination method for the contents of acetic acid in the microspheres.

### Example 12

Preparation of the exenatide-containing microspheres with content (by weight) of the (exenatide acetate+PLGA) and the cleaning solution of 1:400.

Preparation method: Weigh 7.5 g of exenatide acetate and dissolve it in 25 ml of distilled water under stirring to obtain a solution. Dissolve 142.5 g of a copolymer of lactide and glycolide (PLGA 5050 2A 16000) in 675 ml of dichloromethane (CH₂Cl₂) under stirring to obtain another solution. Then, the exenatide acetate solution was mixed with the PLGA solution to obtain a primary emulsion. Subject 75 L of the prepared 1% PVA aqueous solution (see example 3 for the detailed preparation method) to sterile filtration. Then, it was added into a vacuum emulsification blender (referred herein as microspheres preparation kettle) and cooled to 7-13° C. for use as an outer aqueous phase. The primary emulsion was added into the microspheres preparation kettle under a homogenization rate of 400 rpm. Then, it was homogeneously emulsified for 60 s. Then, it was emulsified under a reduced homogenization rate of 150 rpm for 4 h to obtain the solid microspheres, which were then filtered and collected. The microspheres were rinsed with 60 g of distilled water, and then transferred into a freeze-drying tray. An appropriate amount of mannitol injection solution and an appropriate amount of water were added into the freeze-drying tray, and then the freeze-drying tray was placed in a freeze drier to freeze-dry for 36 h at 40° C. (the glass-transition temperature of the mixture of exenatide acetate and PLGA is 43° C.). Then, the freeze-dried product was subjected to sieving and mixing to obtain the microspheres.

See table 4 for the determination method for the content of acetic acid in the microspheres.

### Example 13

Preparation of the exenatide-containing microspheres taking 38° C. as the re-drying temperature.

Preparation method: Weigh 7.5 g of exenatide acetate and dissolve it in 25 ml distilled water under stirring to obtain a solution. Dissolve 142.5 g copolymers of lactide and glycolide (PLGA 5050 2A 16000) in 675 ml of dichloromethane (CH₂Cl₂) under stirring to obtain another solution. Then, the exenatide acetate solution was mixed with the PLGA solution to obtain a primary emulsion. Subject 75 L of the prepared 1% PVA aqueous solution (see example 3 for the detailed preparation method) to sterile filtration, then added into a vacuum emulsification blender (referred herein as microspheres preparation kettle) and cooled to 7-13° C. for use as an outer aqueous phase. The primary emulsion was added into the microspheres preparation kettle under a homogenization rate of 400 rpm. It was homogeneously emulsified for 60 s, and then it was emulsified under a reduced homogenization rate of 150 rpm for 4 h to obtain the solid microspheres, which were then filtered and collected. The microspheres were rinsed with 49.5 Kg of distilled water, and then transferred into a freeze-drying tray. An appropriate amount of mannitol injection solution and an appropriate amount of water were added into the freeze-drying tray, and then the freeze-drying tray was placed in a freeze drier to freeze-dry for 96 h at 38° C. (the glass-transition temperature of the mixture of exenatide acetate and PLGA is 43° C.). Then, the freeze-dried product was subjected to sieving and mixing to obtain the microspheres.

See table 4 for the determination method for the contents of acetic acid in the microspheres.

### Comparative Example 5

Preparation of the exenatide-containing microspheres comprising more than 0.01% acetic acid by weight.

Weigh 7.5 g exenatide acetate and dissolve it in 25 ml of distilled water under stirring to obtain a solution. Dissolve 142.5 g of a copolymer of lactide and glycolide (PLGA 5050 2A) in 675 ml of dichloromethane (CH₂Cl₂) under stirring to obtain another solution. Then, the exenatide acetate solution was mixed with the PLGA solution to obtain a primary emulsion. Subject 75 L of the prepared 1% PVA aqueous solution (see example 3 for the detailed preparation method) to sterile filtration, then added into a vacuum emulsification blender (referred herein as microspheres preparation kettle) and cooled to 7-13° C. for use as an outer aqueous phase. The primary emulsion was added into the microspheres preparation kettle under a homogenization rate of 400 rpm. It was homogeneously emulsified for 60 s, and then it was emulsified under a reduced homogenization rate of 150 rpm for 4 h to obtain the solid microspheres, which were then filtered and collected. The microspheres were rinsed with 30 g of distilled water, and then transferred into a freeze-drying tray. An appropriate amount of mannitol injection solution and an appropriate amount of water were added into the freeze-drying tray, and then the freeze-drying tray was placed in a freeze drier to freeze-dry for 36 h at 38° C. (the glass-transition temperature of the mixture of exenatide acetate and PLGA is 43° C.). Then, the freeze-dried product was subjected to sieving and mixing to obtain the microspheres.

See table 4 for the determination method for the contents of acetic acid in the microspheres.

### Test Example 4

### Determination of the Contents of Acetic Acid in Different Batches of Exenatide-containing Microspheres.

Test method: determine with gas chromatography [Appendix VE, Method 3, of Chinese Pharmacopoeia (second part, 2000 Edition)].

Chromatographic Conditions and System Suitability Test

The column was a 10 meters long capillary column with inner diameter of 0.32 mm, and the inner layer was coated with 0.33 μm of FFAP-CB fused silica. The chromatographic conditions were set as follows:

Injection temperature: 220° C.;

Detector temperature: 250° C.;

Split ratio: 100:1;

The column temperature program was set as follows:

starting at temperature of 50° C., and remained the same for 0.10 minutes;

(1) increasing the temperature at a rate of 30° C./min;

(2) the final temperature of 230° C., and remained for 5 minutes;

(3) Injection volume: 1 μl;

The number of theoretical plates: calculated according to acetate peak and should not be less than 5000. The resolution of acetic acid peak and the internal standard peak should conform to the specifications.

Determination of the correction factor: 1.0 ml of n-hexadecane was precisely weighed and dissolved in 30 ml of dimethylformamide in a 50 ml volumetric flask, diluted to volume and shaken well to be used as an internal standard solution. 625 mg of acetic acid reference was precisely weighed and dissolved in dimethylformamide in a 100 ml volumetric flask, diluted to volume and shaken well before use. Ten (10) ml of the aforesaid solution was transferred into a 100 ml volumetric flask. Five (5) ml of the internal standard solution was added.

The solution was dissolved with dimethylformamide and diluted to volume and shaken well. One (1) μl of the solution was injected into gas chromatography and injected continually for 3-5 times. The correction factor was calculated according to average peak areas.

Test sample preparation and measurement: About 50 mg of exenatide-containing microspheres prepared according to Example 1 was weighed and transferred into a 2 ml volumetric flask, into which 1 ml of dimethylformamide was added to dissolve the sample. One hundred (100) μl internal standard solution was added precisely and then the flask was brought to final volume with dimethylformamide and shaken well. One (1) μl sample was injected into gas chromatograph. The result was calculated by an internal standard method. Results are shown in Table 4.

TABLE 4
Determination of the content of acetic acid in exenatide-containing
micro spheres.

| Test NO. | Content of acetic acid |
|---|---|
| Example 9 | 0.0094% |
| Example 10 | 0.0036% |
| Example 11 | 0.0017% |
| Example 12 | 0.0021% |
| Example 13 | 0.0045% |
| Comparative example 5 | 0.0732% |

Analysis of results: for the microspheres of exenatide or a salt or an analogue thereof prepared in examples 9-13, the microspheres were rinsed with a cleaning solution, and the ratio by weight of the (exenatide acetate+PLGA) to the cleaning solution is 1:250. The re-drying temperature was set lower than the glass-transition temperature of the mixture of exenatide acetate and PLGA, and the contents of acetic acid in the microspheres were always below 0.01%. The same method was used in the comparative examples, the amount of the cleaning solution is lower than 250 times than the amount of (exenatide acetate+PLGA), and the contents of acetic acid in the microspheres were always above 0.01%.

### Test Example 5

### Comparative In Vivo Release Experiment of the Exenatide-containing Microspheres Before and After the Stability Test

Test materials:

Test drugs: the exenatide-containing microspheres in Examples 11 and 13 were studied for stability by conducting in vivo release test between samples immediately after preparation (0 month) and after storage for 6 months at temperature of 40° C. and humidity of 75%. The test results are showed in Table 5.

Experimental animals:
SD rats (Shandong Luye Pharmaceutical Co., Ltd. Animal Room).
Test instruments:
a QTRAP5500 mass spectrometer fitted with an ion-spray ionization source (Applied Biosystem, Inc.);
an Agilent 1290 high performance liquid chromatography system comprising a dual infusion pump, an autosampler and a column oven;
an Anke TGL-16G Feige desk centrifuge, (Shanghai Anting Scientific Instrument Factory); and
a Turbo Vap LV pressure blowing concentrator, (Biotage, Inc).

Test method:

Experimental animals: male SD rats with body weight of 240±20 g, 16 per group;
Route of administration and dose: abdominal subcutaneous injection at a dose of 2 mg/kg.
Blood sampling time: blood samples were collected before (at Oh) and after administration at 1 h, 6 h, 1 d, 2 d, 3 d, 5 d, 7 d, 9 d, 11 d, 14 d, 16 d, 18 d, 21 d, 23 d, 25 d, and 28 d, respectively.
Determination of biological samples: Enzyme linked immunosorbent assays method.
Results are shown in Table 5.

TABLE 5
Blood concentrations of exenatide at different time after intramuscular
injection to rats (ng/mL)

| | | Comparative | | | | |
|---|---|---|---|---|---|---|
| | | example 5 | | Example 11 | | Example 13 |
| | Comparative | (40° C./75%, | Example | (40° C./75%, | Example | (40° C./75%, |
| Time (Day) | example 5 | 6 months) | 11 | 6 months) | 13 | 6 months) |
| 0.042 | 1.16 | 0.74 | 1.16 | 1.25 | 1.07 | 1.05 |
| 0.25 | 0.26 | 0.17 | 0.42 | 0.39 | 0.57 | 0.67 |
| 1 | 0.24 | 0.29 | 0.13 | 0.27 | 0.46 | 0.29 |
| 2 | 0.76 | 1.48 | 1.69 | 0.29 | 0.97 | 1.81 |
| 3 | 1.31 | 3.14 | 1.47 | 2.58 | 4.37 | 3.67 |
| 5 | 3.3 | 3.89 | 5.54 | 4.47 | 4.26 | 4.67 |
| 7 | 6.13 | 3.41 | 9.54 | 9.98 | 10.59 | 9.69 |
| 9 | 8.93 | 5.76 | 5.91 | 6.67 | 4.26 | 5.22 |
| 11 | 4.11 | 1.87 | 2.57 | 1.67 | 1.89 | 1.67 |
| 14 | 1.17 | 1.09 | 1.06 | 1.27 | 1.29 | 1.05 |
| 16 | 0.92 | 0.87 | 0.99 | 0.94 | 0.87 | 0.77 |
| 18 | 0.48 | 0.19 | 0.38 | 0.63 | 0.56 | 0.76 |
| 21 | 0.31 | 0.09 | 0.21 | 0.47 | 0.21 | 0.34 |
| 23 | 0.1 | 0.14 | 0.19 | 0.11 | 0.31 | 0.47 |
| 25 | 0.11 | 0.1 | 0.07 | 0.04 | 0.04 | 0.07 |
| 28 | 0.05 | 0.02 | 0.04 | 0.01 | 0.02 | 0.03 |
| AUC(ng/ml * h) | 1379.75 | 1035.31 | 1427.52 | 1423.07 | 1414.07 | 1426.35 |

The results show that the contents of acetic acid during the manufacturing process of exenatide acetate microspheres have great effects on the bioavailabilities of the products within the storage periods. After the stability test (stored for 6 months at temperature of 40° C. and humidity of 75%), the amounts of in vivo releases for the products did not change when the contents of acetic acid were below 0.01%. The bioavailabilities of the microspheres without control of the contents of acetic acid have declined by more than 20%.

### INDUSTRIAL APPLICATION

The present invention provides exenatide-containing compositions, and the compositions are in the form of microspheres. The exenatide-containing microspheres comprise exenatide acetate and a copolymer of lactide and glycolide (PLGA) as raw materials, and the contents of acetic acid in the prepared exenatide-containing composition are lower than 0.01%.

In the manufacturing process of the microspheres, an appropriate amount of applicable cleaning solution is used, an appropriate re-drying temperature is selected during the freeze-dried process, to obtain a composition of exenatide-containing microspheres with good characteristics in accordance with the quality standard, namely the stability is improved and the product shelf lives are extended. In the manufacturing process of the microspheres, an appropriate outer aqueous phase is used to obtain microspheres with uniform particle size and minimum residue of the polymers, to provide a composition with better quality for the clinical use. The present invention is suitable for industrial applications.

What is claimed is:

1. An exenatide-containing composition, wherein the composition is in a form of microspheres and is prepared with exenatide acetate or another salt or an analogue thereof and a copolymer of lactide and glycolide (PLGA) as raw materials, wherein a content of acetic acid in the exenatide-containing composition is lower than 0.01%, wherein a content of exenatide is 2-10% by weight, and wherein a content of the copolymer of lactide and glycolide (PLGA) is 90-98% by weight.

2. The composition according to claim 1, wherein a weight average molecular weight of the PLGA is 6,000-45,000 Dalton.

3. The composition according to claim 1, wherein a molar ratio of lactide to glycolide in the PLGA is from 90:10 to 10:90.

4. The composition according to claim 2, wherein the PLGA has an intrinsic viscosity of 0.10-0.40 dl/g.

5. The composition according to claim 1, wherein the microspheres are prepared by emulsion-solvent evaporation technique, which includes adding the PLGA into an organic solvent to obtain an organic solution, then adding exenatide acetate into the prepared organic solution to obtain a primary emulsion, then adding the primary emulsion into an outer aqueous phase to form an emulsion, then solidifying and volatilizing the emulsion to remove the organic solvent; and then washing the residue, followed by freeze-drying the residue to obtain the microspheres.

6. The composition according to claim 5, wherein the microspheres are washed with a cleaning solution and then re-dried_in a second freeze-drying process, wherein a weight ratio of microspheres to the cleaning solution is a predetermined ratio, and the microspheres are re-dried at a selected temperature.

7. The composition according to claim 6, wherein the organic solvent is selected from dichloromethane, acetone and acetonitrile, wherein the cleaning solution is distilled water or alkaline buffer, and a weight ratio of microspheres to the cleaning solution is 1:250 and above.

8. The composition according to claim 6, wherein the selected temperature used in the second freeze-drying process is lower than the glass-transition temperature of the mixture of exenatide acetate and PLGA.

9. The composition according to claim 5, wherein the outer aqueous phase is a polyvinyl alcohol solution, wherein a content of the polyvinyl alcohol in the composition is not more than 0.008% by weight.

10. The composition according to claim 9, wherein the content (by weight/volume %; (g/100 ml)) of the polyvinyl alcohol in the outer aqueous phase is 0.5-1.5 w/v %, wherein a pH value of the outer aqueous phase is 5.6±1.0.

11. The composition according to claim 10, wherein the pH value of the outer aqueous phase is adjusted with a water-soluble acid, base or buffer solution, wherein the acid solution is 0.1 mol/L hydrochloric acid aqueous solution, the base solution is 0.1 mol/L sodium hydroxide aqueous solution, the buffer solution is a phthalate buffer that has a pH value of 5.6.

12. The composition according to claim 9, wherein a weight average molecular weight of the polyvinyl alcohol is 13,000-23,000 Dalton.

13. A method for preparing a composition of exenatide-containing microspheres, comprising:
using exenatide or a salt or an analogue thereof and a copolymer of lactide and glycolide (PLGA) as raw materials, and
preparing the microspheres using emulsion-solvent evaporation technique, which comprises:
adding the PLGA into an organic solvent to obtain an organic solution,
then adding exenatide acetate into the prepared organic solution to obtain a primary emulsion,
then adding the primary emulsion into an outer aqueous phase to form an emulsion,
then solidifying and evaporating the emulsion to remove the organic solvent; and
then washing and freeze-drying the residue to obtain freeze-dried microspheres.

14. The method according to claim 13, wherein the washing is with a cleaning solution at a selected ratio, and further comprising re-drying at a selected temperature.

15. The method according to claim 14, wherein the organic solvent is selected from dichloromethane, acetone and acetonitrile, wherein the cleaning solution is distilled water or alkaline buffer solution, and a weight ratio of microspheres to the cleaning solution is 1:250 and above.

16. The method according to claim 14, wherein the re-drying temperature used in the freeze-dried process is lower than the glass-transition temperature of the mixture of exenatide acetate and PLGA.

17. The method according to claim 13, wherein the outer aqueous phase is a polyvinyl alcohol solution, a content (by weight/volume; (g/100 ml)) of the polyvinyl alcohol in the outer aqueous phase is 0.5-1.5 w/v %, wherein a pH value of the solution is 5.6±1.0, and wherein a content of the polyvinyl alcohol in the composition is not more than 0.008% by weight.

18. The method according to claim 17, wherein the pH value of the outer aqueous phase is adjusted with a water-soluble acid, base or buffer solution, wherein the acid solution is 0.1 mol/L hydrochloric acid aqueous solution, the base solution is 0.1 mol/L of sodium hydroxide aqueous solution, the buffer solution is phthalate buffer (pH=5.6).
